(19) United States
(12) Patent Application Publication
(10) Pub. No.: US 2009/0208981 A1
(43) Pub. Date: Aug. 20, 2009

(54) Method for Analysis of Interaction Between Small Molecules and Cells and Apparatus thereof

(76) Inventor: Jia-Ming CHANG, Taipei (TW)

Correspondence Address: Hung-Chih Tseng;HDLS IPR Services, PO Box 220746, Chantilly, VA, 20153, US

(21) Appl. No.: 12/413,597

(22) Filed: Mar. 29, 2009

Related U.S. Application Data
(63) Continuation-in-part of application No. 11/119,908, filed on May 3, 2005, now abandoned.

(51) Int. Cl. G01N 33/567; C12M 1/34

(52) U.S. Cl. 435/7.25; 435/7.21; 435/288.6

(57) ABSTRACT

A method and an apparatus are provided for analyzing the interaction between small molecules and cells without using density gradient and antibody but using a column packed with identical resin particles. The interactions between cell surface and resin particles resulting in the different retention time of cells treating by different small molecules in the column contributed to examining whether the small molecule could interact with cell or not. This invention can also apply small molecule screening, drug screening through examining the retention time by using the column.

### RELATED APPLICATIONS

This application is a continuation-in-part of U.S. patent application Ser. No. 11/119,908, filed on May 3, 2005.

### BACKGROUND OF THE INVENTION

1. Field of the Invention

The invention relates to an apparatus and a method for analysis of the interaction between small molecules and cells without using density gradient and antibody.

2. Description of the Prior Art

In current pharmaceutical studies, researchers wish to find the drug target site on cells so as to understand the action mechanism of drug on cells in order to develop more efficient or safer drugs. In the experimental design, homogeneous cells are used to demonstrate the action mechanism of drugs so as to understand the specific response from a particular cell population to a particular drug, and to identify the effect of a drug on different tissue. Thus, the separation and purification of cells play a highly important role in pharmacological experiments. Providing a unique population of cell means not only to provide a directly observable object, but also to understand the action mechanism of a drug in a particular cell.

There are two kind of conventional methods for separating cells, namely, a method in which a special solution, such as Ficoll cell separation medium or Percoll cell separation medium (Percoll, a particulate silica gel treated with polyvinylpyrolidone (PVP)) was used, and based on the gradient due to centrifugal force or varying proportions of substances added, different kinds of cells can be separated into respective density zones in consistent with their own cell density by virtue of centrifugation. For example, U.S. Pat. No. 6,641,517 disclosed an apparatus capable of producing good gradient. Separation of cells by means of density gradient, however, is a difficult operation and time-consumed. Furthermore, the cell separation medium could be toxic on cells to be separated as well as affect the quality of cells.

Another method for separating cells takes advantage of a specific antibody that can recognize the surface molecule on a cell. This antibody can conjugate covalently or through affinity with a fluorescent substance or substance comprising iron-containing component. In case of using an antibody conjugated with a fluorescent substance, a cell sorter can be used to distinguish a particular fluorescence, and meanwhile, upon rendering cells to be charged, specific cells can be separated under an applied electric field, such as described in U.S. Pat. No. 4,629,687. On the other hand, in case of using antibody conjugated with an iron-containing substance, a magnetic field can be used to separate cells labeled with that antibody. Apparatuses and improved apparatuses developed based on this principle were described in, for example, U.S. Pat. Nos. 5,240,856; 5,684,712; 5,691,208; 5,705,059; 5,711,871 and 6,468,432. However, since these apparatuses screen particular cells using their respective antibody, in addition to the high cost of reagents, the development of antibodies might be a limiting factor and at the same time, the utilization of an antibody may render the cell separated being incapable of being used directly in clinical application.

Alternatively, an approach for separating cells by virtue of surface affinity was proposed by Shibusawa in 1999, in which antibodies bound on the resin comprising a stationary phase or on glass beads were used to separate cells by eluting with a competitive substance in buffer solution. As described in the article published by Shibusawa (Shibusawa (1999) J. Chromatography B 722, 71-88), Sepharose 6B or Chromagel A4 combined with polyethylene glycol (PEG) or polypropylene glycol (PPG) were used as the stationary phase in the separation of cells such as granulocytic leukocyte, monocytic leukocyte or erythrocyte, but not in effective separation of certain sub-populations of mononuclear cells such as, for example, T lymphocyte, B lymphocyte and monocytes.

Datar (U.S. Pat. No. 6,008,040) discloses a device that uses a cascade flow for the separation of cells from a fluid mixture. Datar also teaches a bead column for the separation of cells, reads on a column and packing material (col. 4 lines 24-25, FIG. 5.). Also, Datar discloses that are particles at different layers within the column and the particles have diameters of 6 micrometers, 20 micrometers, 100 micrometers and 200 micrometers (col. 11 lines 4-9 & 50). In addition, said beads can be made from polystyrene or other material (col. 11 lines 5-6). In Datar's example 1-6, it describes a size-gradient separation device that could be use to remove leukocyte from sample efficiently (col. 15). Beads 36 may be formed from 200 μm cross-linked agarose, beads 38 may be form from 20 μm polystyrene, and beads 40 may be 6 μm high-performance silica beads. Further in example 7-14, the affinity-enhance, size-gradient separation depletion to improve depletion levels of lymphocytes, neutrophils, granulocytes and monocytes (col. 17).

Moreover, Datar teaches that the blood is diluted and suspended in a Saline solution with preservatives where the Saline solution is being interpreted as a phosphate buffered Saline (PBS) and Datar teaches using PBS to wash and re-suspend the cells (col. 15 lines 49-51, col. 20 lines 30-31).

Abbot (U.S. Pat. No. 4,290,892) teaches an exchange chromatographic separation of poly-functional compounds. Also, Abbot teaches a column packed with a weak ion exchange composition for separating poly-functional compounds (Abstract, col. 2 lines 4-7), reads on a column and packing material in the column. In addition, Abbot discloses beads diameters are between 5 to 15 microns.

Winther (U.S. Pub. No. 2006/0154234) discloses a standard for an ion-exchange chromatography column that is used in conjunction with cytologyical analysis of a target molecule. Winther also discloses that the beads diameter is between 0.5 to 100 microns and that other particle diameters, such as less than 500 or 200 microns, may be used.

There are several different points in the present invention compare with prior art:

1. Present invention discloses a column filled with identical beads having uniform size homogenously without being formed in any layers. Said uniform size can be selected from 1 to 3 millimeter. The column in the present invention is obviously different from the three different layer column of Datar.

2. According to Datar's specification, the diameter of granulocyte is larger than bead 40 (6 μm), so the column could not let granulocyte pass through. Datar claims a process that is used to remove two of the three target components form fluid mixture and one of said three target components is increased in purity during said separation (claim 10). And said removable two of the three target components are rejection antibodies and leukocytes, and said one of the three target components is red blood cells (claim 11). Datar uses a column which is filled with different layer, size and material beads to separate or filter undesired components from the mixture.

However, all drug-treated cells (or un-treated cells) can completely pass through the column with different retention time in the present invention. Specifically, present invention provides a process/method to analyze the interaction between the small molecules and cells by examining the retention time of drug-treated cells (or un-treated cells) in the column.

3. Abbott discloses that the pH value of elution buffer in weak anion exchange could be 4.4, 2.85, 2.85 and 3 respectively (example II to V). However, much higher or lower pH value (basic or acidic condition) could not be employed in the column for elution of alive cell. Further, it could not be used to analyze the interaction between small molecules and cell surface. In the present invention, the suitable pH is required for the elution of the alive cell, preferred pH 7.4.

In conclusion, it is obviously different in the units of apparatus, the process/method, goal of present invention are obviously different from those of the prior arts. Specifically, the method of present invention is used to analyze the interaction between small molecules and cell surface by examining the retention time of drug-treated cells (or un-treated cells) in the column.

In view of the forgoing, there are many disadvantages associated with the above-described conventional methods. Moreover, the operation time of these conventional methods usually take a time period of 2 to 9 hours, which not only is time-consumed, but also might affect the quality of the cell during separation. Therefore, these conventional methods do not have perfect design and need further improved.

In light of these disadvantages associated with those conventional cell separation methods, the inventor of this application devoted to improve, and finally, after extensive study for many years, has developed successfully the apparatus and method for rapid separation of cells according to the invention.

### SUMMARY OF THE INVENTION

The invention provides an apparatus and a method for analysis of the interaction between small molecules and cells without using density gradient and antibodies, characterized in that, in addition to provide simpler and rapid operation procedure, as well as need not use antibody, a more economic apparatus and method can be realized so as to lower the cost involved in research and development.

The invention provides an apparatus and a method without using density gradient and antibodies, characterized in that it needs neither particular chemical agent for producing density gradient nor antibodies for recognizing antigen such that the interaction between small molecules and cell surface can be analyzed.

The invention also provides an apparatus and a method for rapid separation of cells without using density gradient and antibodies, characterized in that it can offer an apparatus and method for separating rapidly and efficiently sub-populations of mononuclear cells.

The invention provides an apparatus and method for analysis of the interaction between small molecules and cells without using density gradient and antibodies, characterized in that it can be used in the screening of drugs, small molecules and biological molecules so as to provide researchers a more convenient and effective tools for screening of the interaction between small molecules and cell surface.

The invention provides a column that can be used in the analysis of the interaction between small molecules and cells, based on the principle of interaction between cell surface and ionic exchange resin. As shown in FIG. 1, since numerous drugs act on the surface receptor of a cell to modify further the physical properties of the cell surface, different kinds of cells will have its own different retention time in the column in the presence or absence of drug action. Therefore, a column developed based on this principle can be used in drug screening (or small molecule screening).

These features and advantages of the present invention will be fully understood and appreciated from the following detailed description of the accompanying drawings.

### BRIEF DESCRIPTION OF THE DRAWINGS

FIG. 1 is a schematic view showing the operation and the principle of the invention;

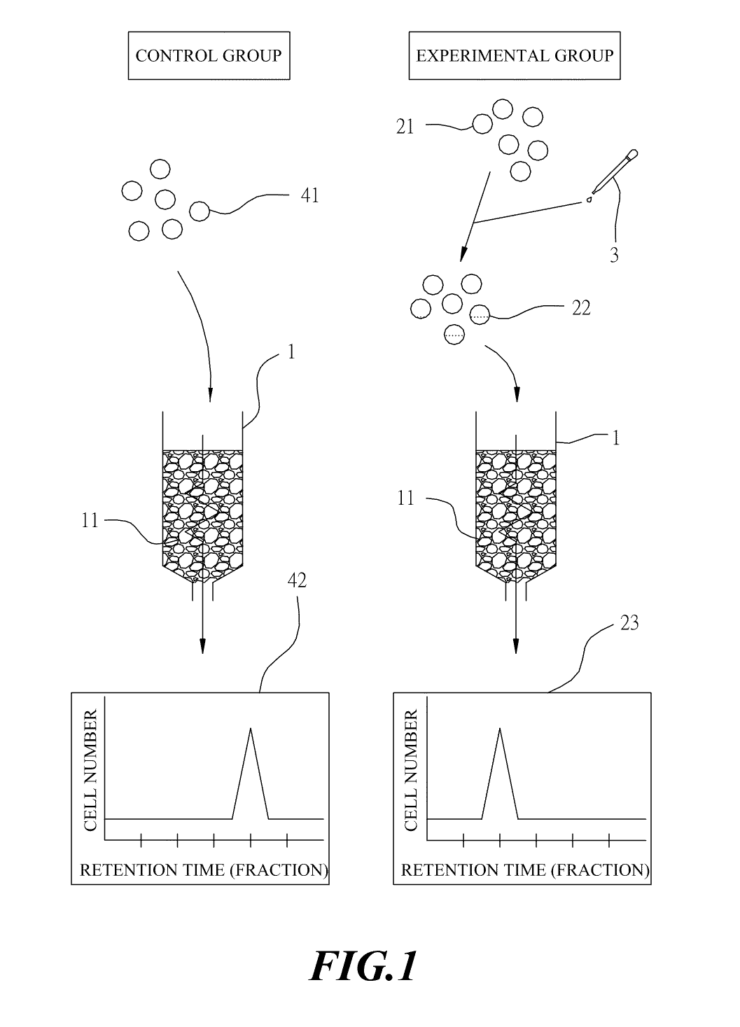

FIG. 2 is a chart showing the number of mononuclear cells collected at different time period during the separation of cells pre-treated with or without lectin on a small column;

FIG. 3 is a chart showing the retention time of PBMC which is altered by the lectin and shikonin in the apparatus of present invention.

FIG. 4 is a chart showing the number of erythrocytes collected at different time period during the separation of erythrocytes-containing mononuclear cells on a small column;

FIG. 5 is a chart showing the number of mononuclear cells collected at different time period during the separation of a concentrated suspension of mononuclear cells;

FIG. 6 is a chart showing the percentage of each sub-population of mononuclear cells collected in the separation on a small column;

FIG. 7 is a chart showing the number of mononuclear cells collected at different time period during the separation of a concentrated mononuclear cells suspension on a large column; and

FIG. 8 is a chart showing the percentage of each sub-population of mononuclear cells collected on a large column.

### DETAILED DESCRIPTION OF THE PREFERRED EMBODIMENT

The apparatus and the method for analysis of the interaction between small molecules and cells without using density gradient and antibody provided according to the invention comprises a column for analysis of the interaction between small molecules and cells, wherein the column can be made of, for example, glass, plastics or metal, and wherein said column is filled with the suitable pH value of buffer for alive cell; in a preferred embodiment, the range of suitable pH value is 6.8-7.6; in a preferred embodiment, the suitable pH value of buffer is phosphate buffered saline (PBS, pH7.4) or serum, and wherein said column can be filled with identical resin particles having uniform size homogenously without being formed in any layer, and said uniform size can be selected from 1 to 3 millimeters, and said identical resin particles can be polystyrene or polyvinyl chloride (PVC), or resin modified with a chemical substance or specific chemical functional group such as, for example, —CN, propyl, phenyl, hydroxylapatite, long chain carbon, NH₃, N,N,N-trimethyl amine (N(CH₃)₃), N,N-diethylamine (N(C₂H₅)₂), or N,N-dimethylamine (N(CH₃)₂) that may be positively charged, and sulfite (SO₃⁻) or carboxyl group (COO⁻) that may be negatively charged. Those chemical substances can be a glycosyl substance such as, for example, a pyranyl, a furanyl, a polysaccharide, or amino acids constituting a protein. The column can take a shape of a cylinder and can be a capillary tube in any size of diameter and length varying as desired. Cells to be analyzed by the apparatus and method for analysis of the interaction between small molecules and cells according to the invention includes, but not limited to: blood cells, peripheral blood mononuclear cell (PBMC), or a suspension of attached cells undergone dissociation.

The small molecule to be analyzed by the apparatus and method for analysis of the interaction between small molecule and cell according to the invention includes, but not limited to: drugs, biological molecules or chemical compounds and the preferred example was lectin and shikonin. Wherein said biological molecules includes, but not limited to: DNA, RNA or protein,

The invention will be illustrated by following non-limiting examples.

### Example 1

### Interaction of Cells and Small Molecules

The invention provides a method and apparatus to screen and analyze the interaction between small molecules and cells. The interactions between cell surface and resin particles resulting in the different retention time of cells treating by different small molecules in the column contributed to examining whether the small molecule could interact with cell or not. In this example, using lectin and shikonin as an analyzed drug (small molecule). Different small molecules may cause different interactions with the same cell, such as binding to the different receptors, or interacting with different cell surface molecules or altering the cell membrane. Hence, this will cause the retention time of the drug-treated cell (or un-treated cell) with the small molecules in the column to be different. According, the method and apparatus can be used to screen the interaction exist or not between small molecules and cell surface.

### 1. Lectin Altered the Retention Time of PBMC in the Cell Chromatography

In this example, the column used for analysis of the interaction between small molecules and cells has an inner diameter of 6 mm, a length of 180 mm and a volume of 5 ml. (180 mm×6 mm) This column was packed with resin particles (size: 1 or 2 millimeter; material: polystyrene) and was washed first and thereafter, filled with a phosphate buffered saline (PBS).

### The Control Group

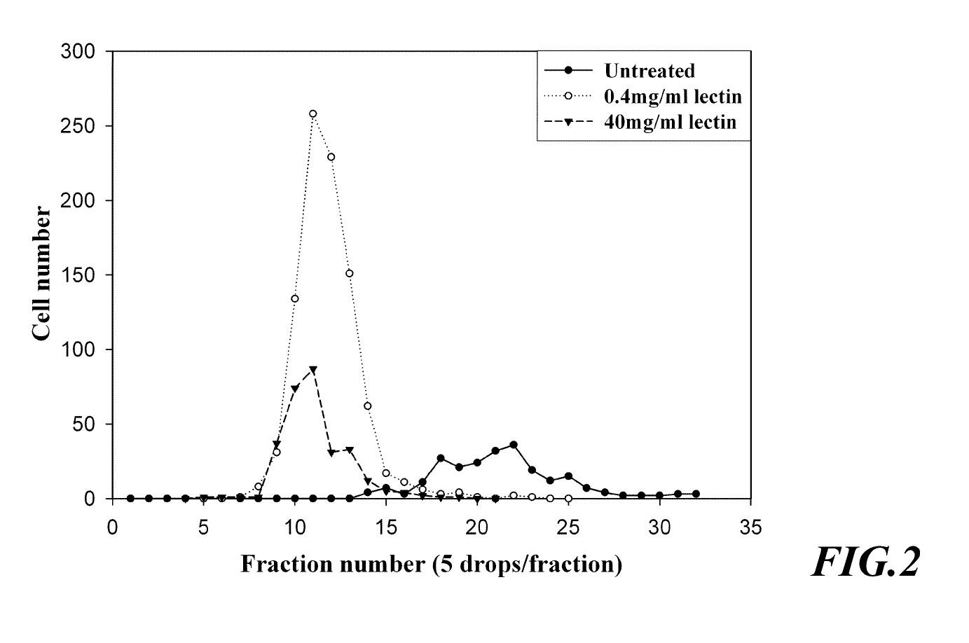

Blood sample was separated first by centrifugation using dextran cell separation medium (Ficoll) to yield peripheral blood mononuclear cell (PBMC). The thus-obtained PBMC was diluted with phosphate buffered saline (PBS) into a cell suspension at concentration of 1×10⁶ cells/ml. This cell suspension was loaded then on the upper frontier of the above-described column. After adding PBS to a level of a pre-determined height, the column was eluted with PBS at a flow rate of 3 ml/min and cell fractions were collected in test tubes, respectively, in a manner that each test tube collected 5 drops of cell suspension eluent. Thereafter, the eluted cell suspension in each test tube was examined under an optical microscope and counted by a cell counter. The result was showed in FIG. 2.

### Experimental Group

Blood sample was separated first by centrifugation using dextran cell separation medium (Ficoll) to yield peripheral blood mononuclear cell (PBMC). The thus-obtained PBMC was diluted with phosphate buffered saline (PBS) into a cell suspension at concentration of 1×10⁶ cells/ml. The cell suspension was added with lectin at a concentration of 0.4 μg/ml or 40 μg/ml. After reacting for 5 minutes, cells thus treated with lectin was loaded on the upper frontier of the above-described column, and after adding PBS to a level of a pre-determined height, the column was eluted with PBS at a flow rate of 3 ml/min and cell fractions were collected in test tubes, respectively, in a manner that each test tube collected 5 drops of cell suspension eluent. Thereafter, the eluted cell suspension in each test tube was examined under an optical microscope and counted by a cell counter. The result was showed in FIG. 2.

As shown in FIG. 2, cells treated with lectin (the experimental group) was eluted out of the column before non-treated cells (the control group), which demonstrated lectin interacted with cells and modified the surface molecules on the cell in a manner that the hydrophilic property of the cell surface was increased, while the interaction with polystyrene was decreased, such that the cell treated with lectin was eluted earlier. Consequently, this method can be used to evaluate and analyze the interaction of cells with chemical molecules, nucleic acids, and proteins.

### 2. Lectin and Shikonin Altered the Retention Time of PBMC in the Cell Chromatography

In this example, the column used for analysis of the interaction between small molecule and cell has an inner diameter of 10 mm, a length of 150 mm and a volume of 11.8 ml (150 mm×10 mm). This column was packed with resin particles (size: 1 or 2 millimeter; material: polystyrene) and was washed first and thereafter, filled with a phosphate buffered saline (PBS).

The method is similar as described as above. Whole Blood was separated first by centrifugation using dextran cell separation medium (Ficoll) to yield peripheral blood mononuclear cell (PBMC). Then PBMC was diluted with phosphate buffered saline (PBS) into a cell suspension at concentration of 1×10⁶ cells/ml.

Assay Group:

Control: un-treated PBMC; twice experiments (control-1 and control-2);

Lectin: treated PBMC by 10 μg/ml lectin for two minutes;

Shikonin: treated PBMC by 10 μg/ml shikonin for two minutes.

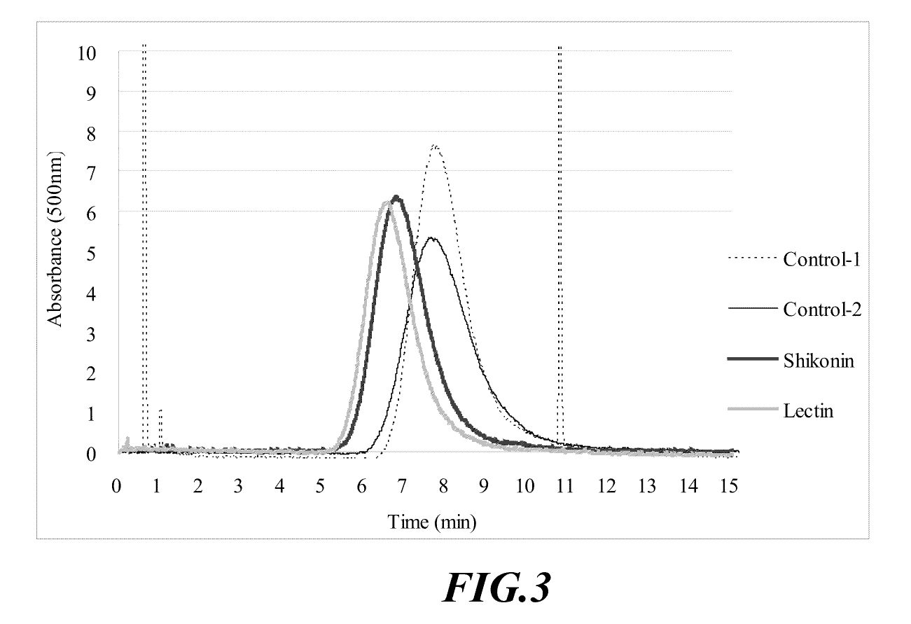

The cell suspension was added with lectin or shikonin at a concentration of 10 μg/ml (lectin treated group or shikonin treated group). After reacting for 2 minutes, drug-treaded group or un-treated group (control) was loaded on the upper frontier of the column (150 mm×10 mm) respectively, and after adding PBS (pH 7.4) to a level of a pre-determined height, the column was eluted with PBS (pH 7.4) at a steady flow rate of 1 ml/min provided under the action of a pump and integrated with a spectrophotometer, which eluted cells were continuously detected with a wavelength of 500 nm. Wherein said spectrophotometer can be replace by flow cytometer. The result was showed in FIG. 3. It shows the retention time of un-treated PBMC (control-1 and control-2) in the column is about 7.7 minutes. The result was identical in two independent experiments (control-1 and control-2). However, the retention time of lectin treated cell or shikonin treated cell in the column was 6.8 minutes and 6.5 minutes, respectively.

It shows cells treated with lectin (or shikonin) was eluted out of the column before un-treated cells (the control group), which demonstrated that lectin (or shikonin) interacted with cells and modified the surface molecules on the cell in a manner that the hydrophilic property of the cell surface was increased, while the interaction with polystyrene (resin particles) was decreased, such that the cell treated with lectin (or shikonin) was eluted earlier. And the retention time of cell in the column could be different depends on different small molecules such as lectin or shikonin. It also indicated the strength of interaction between cells and different small molecules could be different. Hence, the apparatus of present invention could be applied to analyze the strength of interaction contributed by different small molecules.

Specially, present invention provides a method and apparatus thereof to evaluate and analyze the known small molecules, chemical molecules, biological molecules or drugs which could be interacted with the cell surface. Further, the method and apparatus thereof provided by present invention could be applied to the small molecule screening, chemical molecule screening, biological molecular screening or drug screening. According, it helps to screen and validate the unknown small molecules, chemical molecules, biological molecules or drugs that be able to interact with the cell surface. Also, it helps to analyze the strength of interaction between the known molecules and cell surface.

### Example 2

### Separation of Sub-Population of Mononuclear Cells

### Example 2(A)

In example 2(A), the column also could be used for separating cell has an inner diameter of 6 mm, a length of 180 mm and a volume of 5 ml. This column was packed with resin particles and was washed first and thereafter, filled with a phosphate buffered saline (PBS).

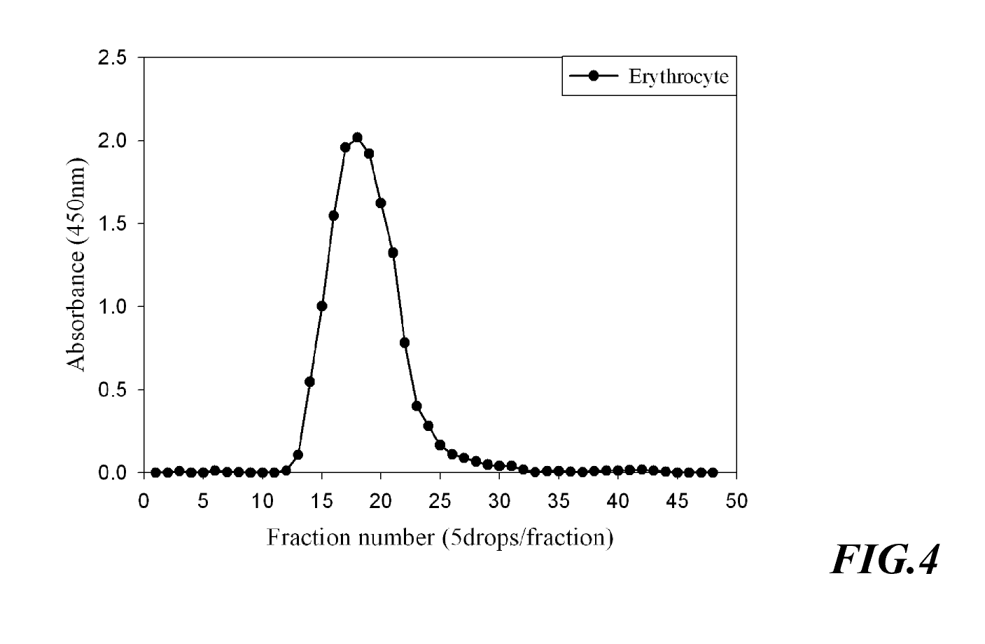

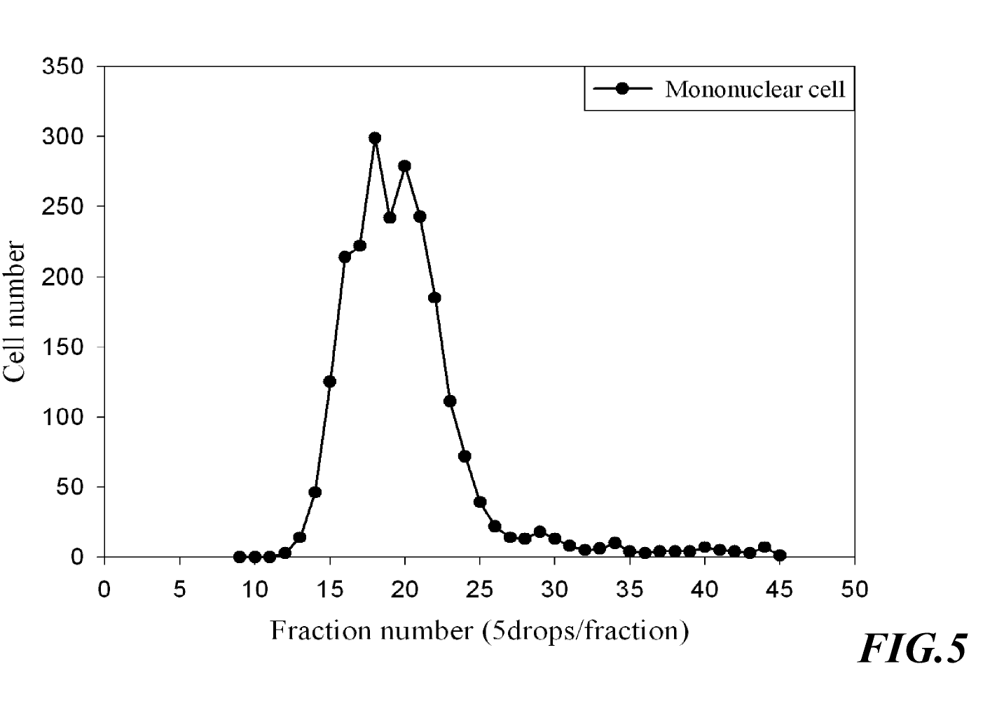

A concentrated mononuclear cells suspension was diluted with PBS to a cell suspension at a concentration of 1×10⁶ mononuclear cells/ml, containing still a certain amount of erythrocytes. This cell suspension was loaded then on the above-described column. The column was eluted subsequently with PBS at a flow rate of 3 ml/min and cell fractions were collected in test tubes, respectively, in a manner that each test tube collected 5 drops of cell suspension eluent. Thereafter, the eluted cell suspension in each test tube was examined under an optical microscope and numbers of erythrocytes and leukocytes were counted by a cell counter, respectively. The results were shown in FIGS. 4 and 5. FIG. 4 shows the number of erythrocytes, while FIG. 5 shows the number of mononuclear cells.

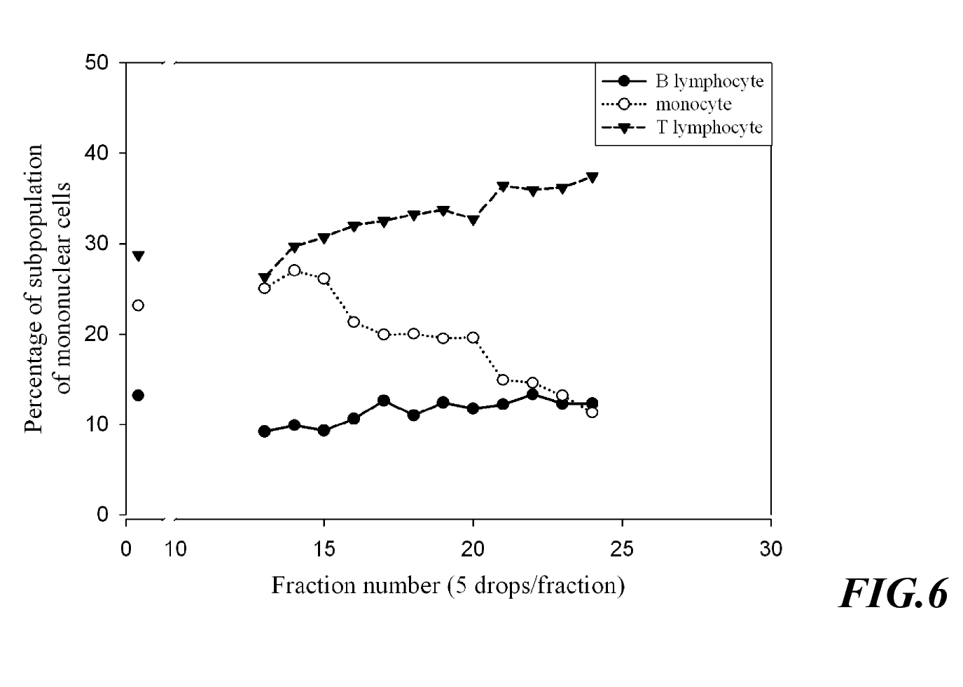

Since each sub-population of mononuclear cells has to be recognized with each own antibody, to the cell suspension eluent in each collecting tube was added 0.1 g of anti-CD3-FITC, anti-CD19-PE and anti-CD14-Cy5 antibodies conjugated with fluorescent substances, in order to recognized T lymphocyte (CD3⁺), B lymphocyte (CD19⁺), and monocyte (CD14⁺), respectively. After carrying out a fluorescent immuno-staining analysis by a flow cytometer, the relative percentages of each sub-population were shown in FIG. 6.

### Example 2(B)

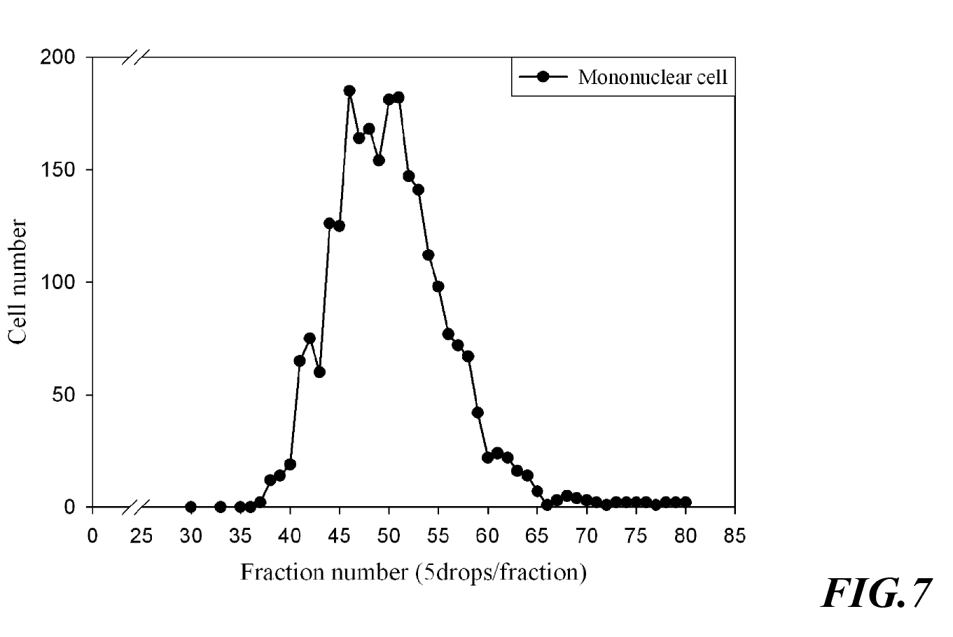

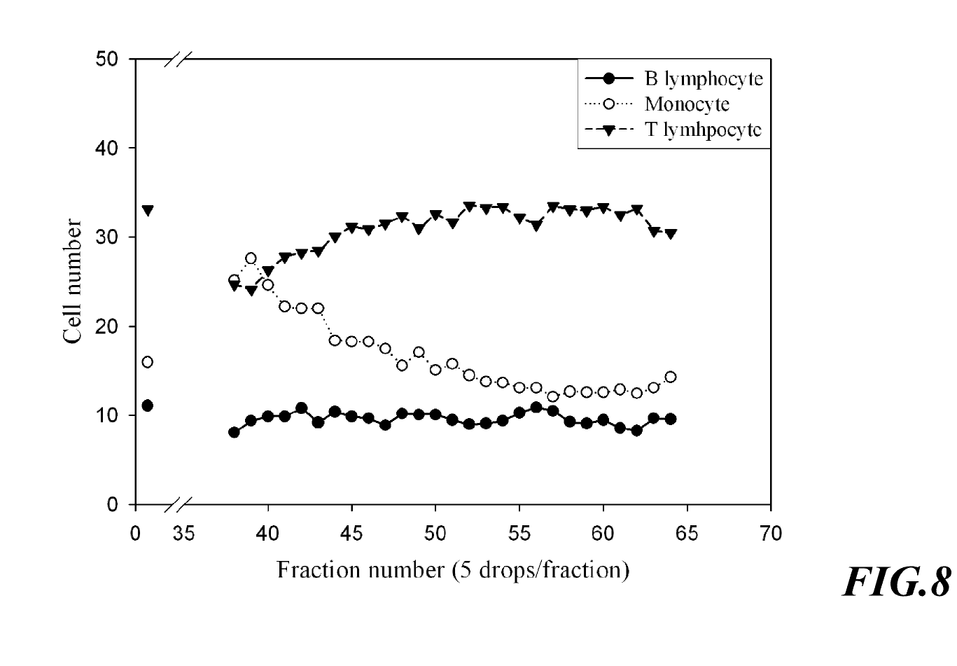

In example 2(B), the column also be used for separating cell was changed and has an inner diameter of 8 mm, a length of 200 mm and a volume of 10 ml. The procedure of example 2(A) was repeated, and the column was filled at a flow rate of 1.2 ml/min. The result was shown in FIGS. 7 and 8. FIG. 7 shows the number of mononuclear cells, while FIG. 8 shows the relative percentage of each sub-population of mononuclear cells.

The results obtained above suggest that the column could not achieve any separation effect against a single population of erythrocyte as shown in FIG. 4. For mononuclear cells in a same sample, several bands were eluted successively, as shown in FIGS. 5 and 7. After analyzing further by fluorescence immuno-staining, the column provided a partition effect with respect to various sub-populations of mononuclear cells such as, T lymphocyte, B lymphocyte and monocyte, and revealed a significantly difference variation, as shown in FIGS. 6 and 8.

The size of the inner diameter and length of the column might have influence on the separation effect and the retention time of treaded-cell (or un-treated cell). The less the diameter of the column is, the better separation effect can be obtained, as demonstrated in FIGS. 6 and 8. For the small column, as shown in FIG. 6, after separating on a small column, T lymphocytes had its percentage increased from 26% (the 13^{th} collection tube) to 39% (the 24^{th} collection tube), which corresponding to a increase of 50% over the original sample, while monocytes had its percentage decreased from 25% (the 13^{th} collection tube) to 10% (the 24^{th} collection tube), which corresponding to a decrease of about 60% over the original sample. Therefore, with this method, by collecting cells eluted at different time period, each sub-population of mononuclear cells can be rapidly and efficiently separated. Obviously, this method can be applied not only for the general separation of blood cells, but also for clinical separation and removal of cancer cells from leukemia patient.

The apparatus and method for analysis of the interaction between small molecules and cells without using density gradient and antibody provided according to the invention has several following advantages over prior patent and conventional techniques recited above:
1. The apparatus and method according to the invention has a simpler and rapid operation procedure to analysis of the interaction between small molecules and cells without using any antibody, and hence provides a more economical apparatus and method to reduce the cost of research and development.
2. The apparatus and method according to the invention can achieve the desired cell separation without using any special chemicals such that the quality of cells thus separated can be assured.
3. The apparatus and method according to the invention can separate rapidly and efficiently each sub-population of mononuclear cells without using any antibody, thereby the cell thus separated can be used directly for further research and development as well as for clinical application.
4. The apparatus and method according to the invention can be used for evaluating and analyzing the interaction of cells with chemical molecules, nucleic acids, and protein, and is applicable further in the screening of drugs.
5. The apparatus and method according to the invention can be used in continuous separation process, and can be used in combination with other analytical instruments in a continuous and timely analytical system.
6. The apparatus and method according to the invention can be applied in the usual separation of blood cells for research use, as well as for the separation and removal of cancer cells from the clinical leukemia patients.

While the above description gives only a specific illustration for an embodiment of the invention, it is understood that the embodiment is not used to limit the scope of the invention. Any equivalent variation and modification not departing from the spirit of the invention are considered to be fallen within the scope of the appended claims.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A method of analyzing the interaction between small molecules and cells, said method comprising the steps of:
(a) cell samples being diluted with a suitable pH value of buffer for alive cell to cell suspension having a pre-determined concentration;
(b) treating the cell suspension obtained from step (a) with the small molecules;
(c) loading the treated-cell suspension or un-treated cell suspension separately into column been packed with identical resin particles having a uniform size, wherein said uniform size is selected from 1 to 3 millimeters, wherein said identical resin particles is selected from the group consisting of polystyrene, polyvinyl chloride (PVC) or the resin particles comprises a chemical substance or a specific chemical functional group, wherein said column filled with the suitable pH value buffer for alive cell;
(d) the cell suspension being eluted with the suitable pH value buffer for alive cell at a constant flow rate, the cell suspension eluent being collected in a test tube in a manner that a collecting test tube is successively replaced at a time interval or after collecting a constant number of cell suspension drop or continuously detected with a spectrophotometer or continuously detected with a flow cytometer;
(e) measuring the retention time of the collected treated cell suspension and un-treated cell suspension to analyzing the interaction between small molecule and cell surface.

2. A method as recited in claim 1, wherein the cell samples are blood cells, peripheral blood mononuclear cell or cell suspensions of attached cells undergone dissociation.

3. A method as recited in claim 1, wherein the small molecule is lectin.

4. A method as recited in claim 1, wherein the small molecule is shikonin.

5. A method as recited in claim 1, wherein the specific chemical functional groups is —CN, propyl, phenyl, hydroxylapatite, long chain carbon, NH3, N,N,N-trimethyl amine (N(CH3)3), N,N-diethylamine (N(C2H5)2), or N,N-dimethylamine (N(CH3)2) that may be positively charged, and sulfite (SO3−) or carboxyl group (COO−) that may be negatively charged.

6. A method as recited in claim 1, wherein the chemical substance is a glycosyl substance or an amino acid.

7. A method as recited in claim 6, wherein the glycosyl substances is a pyranyl substance or a furanyl substance as well as a polysaccharide.

8. A method as recited in claim 6, wherein the amino acids is one of the 20 amino acids constituting a protein.

9. A method as recited in claim 1, wherein the suitable pH value is selected from pH 6.8 to 7.6.

10. A method as recited in claim 1, wherein the suitable pH value of buffer is phosphate buffered saline.

11. A method as recited in claim 1, wherein the suitable buffer is serum.

12. A method as recited in claim 1, wherein the constant flow rate is a flow rate achieved under naturally dropping or a steady flow rate provided under the action of a pump.

13. A method of analyzing the interaction between small molecules and cells, said method comprising the steps of:
(a) cell samples being diluted with phosphate buffered saline or serum to cell suspension having a pre-determined concentration;
(b) treating the cell suspension obtained from step (a) with the small molecules;
(c) loading the treated-cell suspension or un-treated cell suspension separately into column been packed with identical resin particles having a uniform size, wherein said uniform size is selected from 1 to 3 millimeters, wherein said identical resin particles is polystyrene or polyvinyl chloride (PVC), wherein said column filled with phosphate buffered saline or serum;
(d) the cell suspension being eluted with phosphate buffered saline or serum at a constant flow rate, the cell suspension eluent being collected in a test tube in a manner that a collecting test tube is successively replaced at a time interval or after collecting a constant number of cell suspension drop or continuously detected with a spectrophotometer or continuously detected with a flow cytometer;
(e) measuring the retention time of the collected treated cell suspension and un-treated cell suspension to analyzing the interaction between small molecule and cell surface.

14. A method as recited in claim 13, wherein the small molecule is lectin.

15. A method as recited in claim 13, wherein the small molecule is shikonin.

16. An apparatus for analyzing the interaction between small molecule and cell without using density gradient and antibody, comprising a column and the identical resin particles having uniform size packed in the column, wherein said uniform size of resin particle is selected from 1 to 3 millimeters, wherein said column is filled with the suitable pH value buffer for alive cell.
